(19) United States
(12) Patent Application Publication
(10) Pub. No.: US 2018/0165424 A1
(43) Pub. Date: Jun. 14, 2018

(54) METHOD FOR DYNAMIC BIAS MANAGEMENT BETWEEN ONLINE PROCESS ANALYZERS AND REFEREE TESTS

(71) Applicant: ExxonMobil Research and Engineering Company, Annandale, NJ (US)

(72) Inventors: Kevin R. Worden, Minooka, IL (US); Tian C. Lau, Whitby (CA); Kathryn L. Hackman, Glen Ellyn, IL (US); Sean R. Werner, Woodridge, IL (US); David L. Rogers, Plainfield, IL (US); Clifton G. Hene, Aurora, IL (US)

(21) Appl. No.: 15/830,498

(22) Filed: Dec. 4, 2017

Related U.S. Application Data
(60) Provisional application No. 62/433,847, filed on Dec. 14, 2016.

(51) Int. Cl. G06F 19/00; G01N 33/28

(52) U.S. Cl. CPC G06F 19/703; G01N 33/287; G01N 33/2852

(57) ABSTRACT

Provided herein are methods for dynamic bias management between online process analyzers and laboratory certification tests. In many refinery processes, online analyzers are used to determine any number of properties to ensure the product being produced meets a given target or specification. Refinery laboratory tests are typically used for certification, and therefore, biases between said certification tests and online process analyzers need to be managed to control/optimize the manufacturing process. The methods employ an exponential-weighted moving average (EWMA) dynamic correction factor based on historical process analyzer data versus laboratory certification samples in conjunction with structural bias correct functions to achieve this bias management.

### CROSS-REFERENCE TO RELATED APPLICATIONS

This application claims priority to U.S. Provisional Application Ser. No. 62/433,847 filed Dec. 14, 2016, which is herein incorporated by reference in its entirety.

### FIELD

The present disclosure relates to a method for dynamic bias management between online process analyzers and referee test methods. Specifically, the method relates to assuring that online to process analyzer outputs match referee tests results used for certification with a high degree of certainty for a given property of refinery product.

### BACKGROUND

In the operation of refinery processes (e.g. hydrotreaters, gasoline blenders, etc.) online process analyzers are typically used to determine the quality of a property of the product resulting from the process, such as octane, Reid vapor pressure (RVP), temperature at which the vapor to liquid ratio is 20:1 (TV/L (20)), distillation boiling points, sulfur content, aromatic content, olefin content, etc. That quality is measured against a target value or specification. Standard laboratory tests, referred to herein as “laboratory certification tests,” “validation tests,” or “referee tests” are typically the mandated tests used to certify that the product meets the required specifications. Accordingly, the biases between the online process analyzer outputs and referee test results need to be established and continuously validated to ensure operator confidence in the use of online process analyzers to control and optimize the refinery process at issue.

As is well understood, discrepancies between online process analyzers and laboratory tests, known as bias, can have serious economic consequences. If the online process analyzer indicates the product is within specification, but that same product fails the referee test, then the product must be down-graded or reblended to meet specifications before it is marketable. Alternatively, if the bias between the online process analyzer and the referee test method is such that it results in overshooting the specifications, valuable resources are potentially wasted (referred to herein as “giveaway” or “quality giveaway”).

Conventional bias management is accomplished in a variety of ways. In the most basic process, analyzer bias is manually adjusted by applying a constant correction term to the raw analyzer value and can be represented by the equation:

Corrected Analyzer Output=Raw Analyzer Output+Bias Correction

However, a constant bias is typically ineffective because the bias between two methods often changes over time due to a variety of reasons such as process changes, range of the property, and analyzer drift.

Another common bias correction method, referred to as full bias correction, uses samples obtained from the process (validation sample) to periodically update the analyzer bias. The difference between the lab referee test result and the analyzer output at the sample time is called the residual. This residual from the most recent validation sample is applied to the current raw analyzer output each time a validation sample is obtained from the process. This full bias correction can be represented by the equation:

Corrected Analyzer Output=Raw Analyzer Output+Analyzer/Lab Residual

Due to the inherent variability in all analytical test methods, this method adds additional variability to the Corrected Analyzer output.

ASTM Standard Practice D6708 is also commonly used to determine a bias correction function, where there is a linear relationship between the two methods over a given range, which is represented by the equation:

Corrected Analyzer Output=A*Raw Analyzer Output+B

Where A and B are empirically determined coefficients. This linear bias correction does not account for short term deviations between the two methods often referred to as autocorrelation.

Additional bias correction functions can be continuous functions (e.g. polynomials) or discontinuous functions (e.g. piecewise linear) obtained from regression analysis techniques. Similarly, these bias correction methods fail to account for autocorrelation between the two analytical methods due to short term deviations.

The conventional bias correction methods described above fail to account for multiple factors that can cumulatively contribute to bias inaccuracies between online process analyzers and laboratory instruments. For example, current methods fail to recognize the need to reduce noise, or variation in the difference between analyzer output and laboratory results on validation samples, to improve confidence in bias corrected online process analyzer output. Additionally, current methods do not provide a means to automatically adjust the bias applied to process analyzer output when it is not aligned with referee test results due to short term deviations. Current methods further fail to account for bias shifts related to product composition (matrix effects) or to process unit operational changes. Also, current methods do not detect when statistically and operationally significant bias shifts have occurred.

There is a need for a dynamic bias correction method to provide improved situational awareness, process control, property target optimization, and confidence in product certification results when using online process analyzers. The present method employs an exponentially-weighted moving average (EWMA) dynamic correction factor that greatly reduces bias and can reduce variation between online process analyzers and laboratory referee test results.

Additionally, conventional (oxygenate-free) mogas (gasoline sold at the pump for road use) has been largely replaced by ethanol-containing gasoline in the United States, Canada, Europe and other countries are also mandating the use of oxygenates in gasoline. Currently, alcohols are favored to supply the mandated levels of oxygen in the blended fuels as environmental problems have arisen with respect to other oxygenates such as ethers. Ethanol is the alcohol most frequently used in view of its economics and availability from agricultural sources.

As explained in U.S. Pat. No. 6,258,987 (Schmidt), the ethanol is not usually blended into the finished gasoline within the refinery because the ethanol is water soluble. As a consequence of this solubility, an ethanol-containing gasoline can undergo undesirable change if it comes in contact with water during transport through a distribution system, which may include pipelines, stationary storage tanks, rail cars, tank trucks, barges, ships and the like: absorbed or dissolved water will then be present as an undesirable contaminant in the gasoline. Alternatively, water can extract ethanol from the gasoline, thereby changing the chemical composition of the gasoline and negatively affecting the specification of the gasoline, possibly leading to regulatory violations since the government may require a certain oxygenate content in the gasoline sold at the pump. Government regulation in the U.S., for example, has until recently limited the oxygen content of gasoline to 4.0 wt. % while also requiring that reformulated gasolines contain at least 1.5 wt. % of oxygen, resulting in the gasoline known as E10 when ethanol is used as the oxygenate at nominally 10 vol %. More recent regulations propose a grade known as E15 for newer vehicles and other grades are also on sale, for example, E85, for use in multi-fuel engines.

In order to avoid contact with water as much as possible, ethanol-containing gasoline is usually manufactured by a multi-step process in which the ethanol is incorporated into the product at a point which is near the end of the distribution system, e.g. at the product distribution terminal, “at the rack”. More specifically, gasoline which contains a water soluble alcohol such as ethanol, is generally manufactured by producing an unfinished and substantially hydrocarbon precursor subgrade or basestock usually known as a Basestock for Oxygenate Blending (BOB) at the refinery, transporting the BOB to a product terminal in the geographic area where the finished gasoline is to be distributed, and mixing the BOB with the desired amount of alcohol at the terminal.

As ethanol is typically blended at the distribution terminal and not at the refinery gasoline blend header, problems arise in the operation of the overall manufacturing and distribution process. Ethanol-free gasoline is typically produced within a refinery as a finished product which fully meets all necessary specifications for sale as an ethanol-free product. This finished gasoline can be manufactured to fit the required product specifications very precisely because analytical data for the product can be obtained during the manufacture (aka gasoline blending) process and used to control the blending process. As a consequence, manufacturing costs are kept to a minimum because expensive blendstocks are usually not wasted by exceeding specifications. Unfortunately, this type of precise manufacturing control is not possible for blending configurations where the to final commercial grade ethanol-containing gasolines are prepared by mixing a non-ethanol containing subgrade blend manufactured at a refinery with ethanol at a location remote from the refinery.

When an additive, such as an oxygenate, is added to a hydrocarbon basestock the effect on the properties can be mathematically modeled as a structural bias correction function, and is called an oxygenate additive boost function. The method described herein can also be applied to this oxygenate additive boost function. The EWMA dynamic correction factor can be applied to the oxygenate additive boost function to correct for short term deviations between the predicted property result and the laboratory referee test result on the BOB sample, when blended with the additive.

The method for dynamic bias correction described herein can be applied to a number of refinery product properties provided that the property in question is measured by online analyzer and subject to certification by a referee test in a laboratory. Such properties include, but are not limited to, RVP, TV/L (20), sulfur content, aromatic content, olefin content, benzene content, distillation, octane, and API gravity. The bias can also be applied to distillate blending properties such as flash point, kinematic viscosity, cetane, cloud point, and pour point. The online analyzers and respective referee tests for these properties are laid out in detail below.

### SUMMARY

Provided herein are methods for reducing bias between an online process analyzer measurement of a property of a hydrocarbon stream and a laboratory certification test result of the same property. In certain aspects, the methods comprise obtaining a physical sample from the hydrocarbon stream at time t; analyzing the physical sample via a laboratory certification test to obtain a laboratory certification test result for the property of the hydrocarbon stream; providing an online process analyzer to measure the property of the hydrocarbon stream; the online process analyzer obtaining a raw value of the property (“the raw analyzer output”); establishing an exponential-weighted moving average (EWMA) dynamic correction factor for the property measured by the online process analyzer, wherein establishing the EWMA dynamic correction factor comprises; collecting historical values of the property from a plurality of past laboratory certification tests; determining a structural bias correction function for the property by performing a regression analysis on the historical values; establishing a lambda weight, λ, between 0 and 1; calculating EWMA via the following equation:

EWMA_{current}=λ*(LRₜ−BCf(RAₜ))+(1−λ)*EWMAₚᵣᵢₒᵣ, where:
EWMA_{current}=Current exponential-weighted moving average dynamic correction factor;
λ=lambda weight;
LRₜ=the laboratory certification test result of the property of the physical sample at time t;
BCf(RAₜ)=the structural bias correction function applied to the raw analyzer output of the property at time t; and
EWMAₚᵣᵢₒᵣ=the most recently prior calculated EWMA;

correcting the raw analyzer output via the following relationship:

CAₜ=BCf(RAₜ)+EWMA_{current},

where CAₜ is the Corrected Analyzer output at time t; and comparing the Corrected Analyzer output at time t to the laboratory certification test result at time t to determine the bias between the two values. In certain aspects, lambda weight is 0.8 or greater.

In other aspects the methods further include establishing an EWMA high limit and an EWMA low limit via the following equation:

EWMA_{Hi/Low}=+/−k₁*Stdev of Residuals, where:
k₁=any number between 2 and 4;
Residuals=LRₜ−CAₜ; and
Stdev=Standard Deviation

comparing the EWMA_{current} to EWMA_{Hi} and EWMA_{Low}; and adjusting the EWMA to the EWMA_{current} is calculated value of EWMA_{current} if between EWMA_{Hi} and EWMA_{Low}. In yet another aspect, if EWMA_{current} is greater than EWMA_{high} or is less than the EWMA_{low} limit, then EWMA_{current} is set to the EWMA_{high} limit or the EWMA_{low} limit depending on which limit is violated.

Additionally, the methods disclosed herein may also include establishing an EWMA hihigh limit and an EWMA lolow limit via the following equation

EWMA_{HiHigh/LoLow}=+/−12*Stdev of Residuals, where:
k₂=any number between 5 and 8;
Residuals=LRₜ−CAₜ; and
Stdev=Standard Deviation

If EWMA_{current} violates the established EWMA_{HiHigh} limit or the established EWMA_{LoLow} limit, then EWMA_{current} is rejected and the Raw Analyzer output continues to be corrected using EWMAₚᵣᵢₒᵣ.

The multiplier k₁ is typically any number between 2 and 4, inclusive, e.g. 2 or 3 or 4. The multiplier k₂ is typically any number between 5 and 8, inclusive, e.g. 5, 6, 7, or 8. Neither k₁ nor k₂ need be a whole number. In some cases, k₂=2*k₁

The methods disclosed herein, can be used to help bias correct a number of properties measure in refinery operations. Some example properties include Reed Vapor Pressure, TV/L(20), sulfur, aromatic content, olefin content, benzene content, distillation points, octane, API gravity, flash point, kinematic viscosity, cetane level, cloud point, or pour point.

In one aspect, the hydrocarbon stream is a basestock for oxygenate blending (BOB). In another aspect, the obtaining a physical sample from the hydrocarbon stream occurs before the hydrocarbon stream is blended with an oxygenate additive and the laboratory certification test occurs after the physical sample is blended with the oxygenate additive. In some cases described above, the oxygenate is ethanol.

### DRAWINGS

FIG. 1 is a flow chart depicting an embodiment of method preparation and the method itself

FIG. 2 is a graphical depiction of dynamic bias correction of TVL when blending Blendstock for Oxygenate Blending (BOB) gasoline compared to conventional bias correction methods.

FIG. 3 is a graphical depiction of dynamic bias correction of TVL when blending ethanol boosted gasoline compared to conventional bias correction methods.

FIG. 4 is a graphical depiction of the EWMA dynamic correction factor over time which is operating within the EWMA Hi/Lo limits.

### DETAILED DESCRIPTION

The present method uses statistical process control and time series techniques for managing bias and reducing variation between online process analyzer outputs as compared to laboratory test results measuring the same property. The method uses an exponential-weighted moving average (EWMA) dynamic correction factor, calculated using historical online process analyzer data as compared to laboratory validation samples (also referred to herein as “referee samples”) measuring the same properties. The bias data can be updated with each referee sample, which are taken as frequently as required for the product and property, typically several times per day.

As used herein, a process analyzer is a piece of analytical instrumentation or equipment (and supporting ancillary equipment) that measures a compositional component or property of a petroleum product. An online process analyzer is designed to receive a continuous feed of petroleum product during the manufacturing process and provide frequent analysis of its properties.

The frequent data generated by the online process analyzer(s) is used as input to a process control program or is provided to process operators for the purpose of process monitoring. Operators may use this data to determine whether or not adjustments to key manufacturing process operating parameters should be made. Some changes are made to drive the measured property value to an optimal target value using an optimal mixture of components, while ensuring compliance to regulatory and contractual requirements and specifications. Examples of key manufacturing process operating parameters can be (but are not limited to) the relative ratios of component flows to a blending facility, the operating temperature or pressure of a treatment facility (e.g., sulfur removal), or the flow of chemical additives.

More than one online process analyzer may be used for a specific manufacturing process, and throughout the specification any reference to an online process analyzer means one or more online process analyzers. No particular process analyzers are required for implementing the method of the current invention. Process analyzers known and used by those skilled in the art can be used as the process analyzer for analyzing the representative sample. The specific choice of process analyzers will depend on the specific regulatory or contractual requirements for the manufactured petroleum product.

Specifically, the online process analyzers used in manufacturing will depend on the specific composition or property of interest. If there are targets for more than one property or component, it may be necessary to have more than one process analyzer. At least two general classes of analyzers may be used to implement the current invention. Other classes may also be used; but these general classes represent analyzers that are commonly used by those in the art. The first class of analyzers are those that directly measure the composition or property parameter and includes, but is not limited to: RVP analyzers for volatility; physical distillation analyzers for single boiling point or full boiling curve; x-ray or ultraviolet fluorescence analyzers for sulfur; and gas chromatographic (GC) analyzers for measuring distillation; benzene; aromatics; olefins and/or oxygenates. The second class of analyzers are those that use multivariate chemometric models to relate the measured analyzer data (typically a spectrum) to the composition or property parameters of interest, including, but not limited to: gas chromatographic; mass spectrometric; infrared techniques such as Fourier Near Infrared (FTNIR); Raman; and nuclear magnetic resonance (NMR) analyzers.

Standard ASTM methods are the most common laboratory referee tests that are used to bias correct online process analyzers measuring properties of gasoline directly or indirectly using multivariate chemometric models (e.g. FTNIR). A list of common ASTM methods and the properties measured is provided in Table 1 below:

TABLE 1

| | Referee | |
|---|---|---|
| | Test | Examples of Online |
| Property | (ASTM) | Analyzer Methods |
| RVP | D5191 | Automated piston and chamber with |
| | | an integrated pressure transducer. |
| | | Chemometric methods. |
| T V/L (20) | D5188 | Evacuated chamber and piston. |
| | | Chemometric methods |
| Sulfur | D2622 | X-ray fluorescence |
| Distillation | D86 | Gas chromatography. |
| (T10, T50, T90, | | Chemometric methods |
| E200, E300) | | |
| Aromatics/Olefins | D1319 | Gas chromatography. |
| | | Chemometric methods |
| Benzene | D3606 | Gas chromatography. |
| | | Chemometric methods |
| Research Octane/ | D2699/ | D2885, online knock engines. |
| Motor Octane | D2700 | Chemometric methods |
| API Gravity | D1298 | Oscillating U tube. |
| | | Chemometric methods |
| Flash Point | D93 | Flash chamber with spark ignition. |
| | | Chemometric methods |
| Kinematic | D445 | Capillary viscometer. |
| Viscosity | | Chemometric methods |
| Cetane | D613 | Chemometric methods |
| Cloud Point | D2500 | Sample cooling system with optical |
| | | detector. Chemometric methods |
| Pour Point | D97 | Sample cooling system with optical |
| | | detector. Chemometric methods |

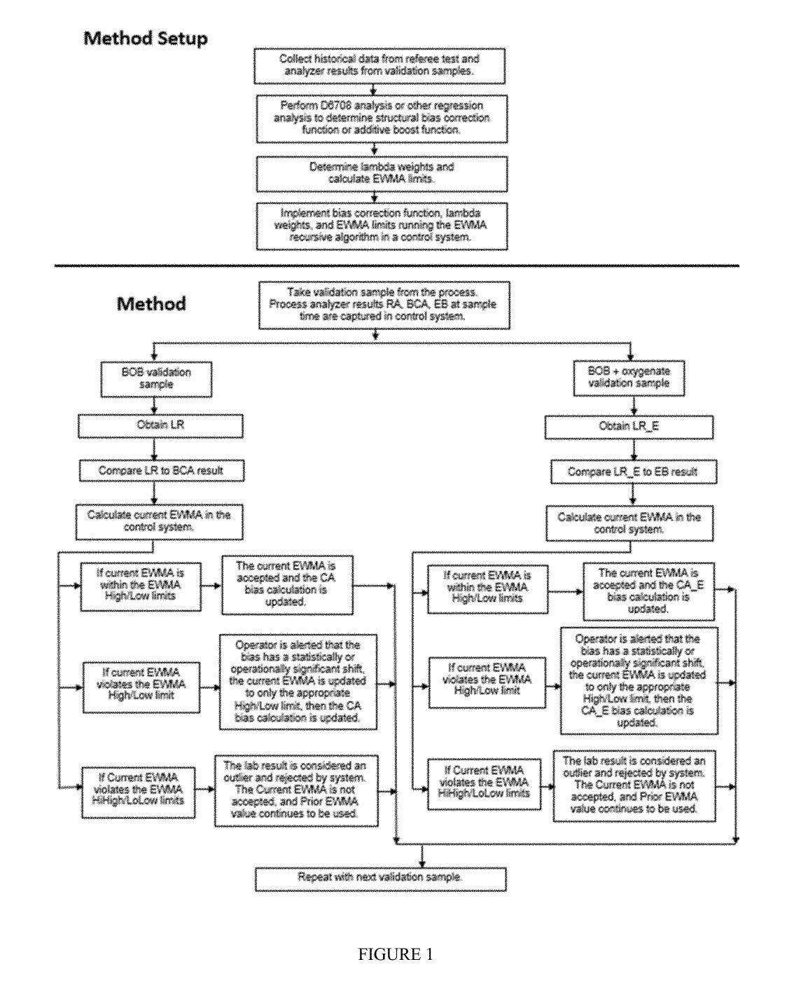

A flow chart of an embodiment of the method is provided in FIG. 1. As shown, there are a few steps required to set up the method. In order to practice the method, one must first calculate the initial structural bias correction function. This involves collecting historical data from past referee test results on validation samples and the corresponding online process analyzer data. Statistically speaking, it is preferred that data from at least 20-40, e.g. 30, samples should be included in the historical data set across the operating range of the property, typically segregated by grade and season. A structural bias correction function is developed to correlate the analyzer results to the referee test results, preferably employing the statistical methodology of ASTM D6708 or other acceptable regression analysis. The structural bias function can be continuous or discontinuous, but will generally be linear. The EWMA correction factor can also be applied to an inferential analyzer for desired properties, such as octane boost from addition of ethanol, or other oxygenate, to a BOB blended gasoline. In this case, the structural bias correction function is the oxygenate boost function, to be described more below.

In order to minimize bias between online process analyzer values and referee test values, an EWMA dynamic correction factor is added to the structural bias correction function, which is applied to the raw analyzer output. The Corrected Analyzer output can be represented by the equation:

CA=BCf(RA)+EWMA_{current} (Eqn. 1)

where CA is the Corrected Analyzer output, BCf( ) is the structural bias correction function, RA is the raw analyzer output, and EWMA_{current} is the current EWMA.

Residuals are the differences between the referee test results and the analyzer results for a given sample and can be represented by the equation:

Residual=LRₜ−Analyzer outputₜ (Eqn. 2)

where LR is the laboratory result of a property for a validation sample at time t and Analyzer outputₜ is the analyzer output at time t, where the analyzer output can be Raw Analyzer output, structural bias correction function, or the Corrected Analyzer output. The standard deviation of the residuals (Stdev) is calculated using historical data and used to determine system variation and to set limits used in the method.

The EWMA dynamic correction is used to correct for short term bias shifts and variation due to normal changes in the system such as calibration of online analyzers or lab instruments, replacement of components in either online analyzers or lab instruments, analyzer drift, using different online analyzers or lab instruments, changes in the process environment, or changes in the matrix composition (e.g. unit startups, blend recipe composition, and/or component quality changes).

As the name implies, the EWMA dynamic correction is frequently checked and/or updated based on a recursive algorithm, typically several times per day, e.g. every 2 hours, every 4 hours, or every 6 hours. The recursive algorithm is given by the following equation:

EWMA_{current}=λ*(LRₜ−BCf(RAₜ))+(1−λ)*EWMAₚᵣᵢₒᵣ (Eqn. 3)

where EWMA_{current} is the current EWMA, λ is weighting factor, LRₜ is the laboratory measurement of a validation sample at time t; BCf (RAₜ)) is the structural bias correction function applied to the raw analyzer output of the at least one property at time t; and EWMAₚᵣᵢₒᵣ is the most recently prior calculated EWMA.

Lambda, λ, is a weighting factor that is between 0 and 1, and typically between 0.1 and 0.9. A lambda of greater than 0.5 means that the most recent referee test result will have a larger impact to the analyzer bias update compared to all prior lab validation test results. A lambda of 0.5 puts an equal weight on the most recent referee test results compared to all prior lab validation test results. Lambda is typically tuned to minimize the StDev of residuals (i.e. LRₜ−BCf(RAₜ)) using the historical data discussed in above. A lambda greater or equal to 0.8 is typically used to aggressively bias correct the analyzer when there is a known change in the composition of the product (e.g. new blend recipe). There are also instances where it would be advantageous to make λ less than 0.5, for example, if there were reason to put less value on a particular online analyzer's measurements.

The current EWMA, as calculated by Eqn. 3, is then compared to certain High/Low limits, which are calculated from the standard deviation (Stdev) of the residuals, represented by the equation:

EWMA Hi/Lo=+/−k*Stdev of Residuals (Eqn. 4)

where k is a multiplier between 2 and 4, typically a value of 3 is used to ensure the change in the EWMA is statistically significant with very high probability of confidence, indicating a bias shift between the analyzer and referee test has occurred. Using a multiplier of 3, there is a probability of 3 out of 1,000 chance of randomly violating the Hi/Lo limit, assuming the residuals are normally distributed. Additionally, higher high and lower low limits, known as EWMA HiHigh/LoLow limits are calculated using the same Eqn. 4, but k is a multiplier between 5 and 8, typically a value of 6. In any case, it is preferable that the multiplier for EWMA HiHigh/LoLow is twice the multiplier for EWMA Hi/Lo (e.g. 2 k and k, respectively). These limits are used to flag when the EWMA_{current} is a statistical outlier, and therefore the current analyzer output is not updated with this EWMA_{current}. Using a multiplier of 6, there is a very small probability (less than 1 out of 1,000,000 chance) of violating the HiHigh/LoLow limit randomly, assuming the residuals are normally distributed.

As discussed above, analyzer output is corrected using Eqn. 1. The EWMA update to its current value takes into account short term bias shifts and variation due to changes such as; calibration of online analyzers or lab instruments, instrument part replacement, analyzer drift, using back-up lab instruments, changes in the process or environment, or changes in the matrix composition (e.g. unit startups, blend recipe composition, and/or component quality changes).

Operation of the method can be explained with reference to FIG. 1. First, the operator takes a validation sample from the process. At the sample time, the raw analyzer output (RA), and the bias Corrected Analyzer output (BCA) are captured in the control system. Referee test results are obtained from the validation sample for the appropriate properties. The referee test results are then compared to the bias Corrected Analyzer output.

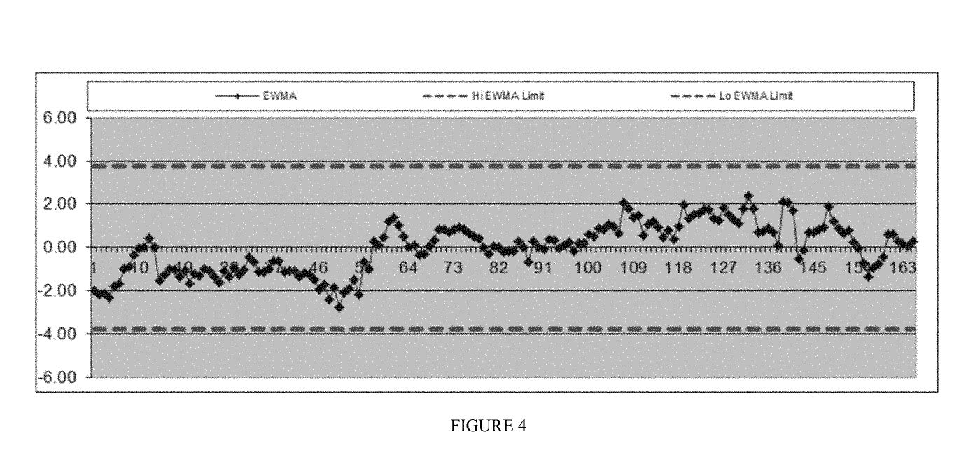

Next, EWMA_{current} is calculated using Eqn. 3. If EWMA_{current} is within the EWMA High/Low limits, then the newly calculated EWMA_{current} is accepted by the control system and the CA bias calculation is updated in the control system. If EWMA_{current} violates the EWMA High/Low limit, then the operator is alerted that the bias has had a statistically or operationally significant shift. In this case, the EWMA_{current} is updated only to the applicable EWMA High/Low limit (i.e. EWMA_{current}=EWMA High/Low limit), and then the CA bias calculation is updated. Operation of the EWMA as compared to the EWMA Hi/Lo limits is shown in FIG. 4. The EWMA_{High} and EWMA_{Low} are set at about 3.8 and −3.8 respectively in this example. The x-axis shows EWMA over time as calculated using Eqn. 3. Each diamond on the x-axis represents a different spot sample subjected to a laboratory referee test.

There are several scenarios when EWMA_{current} could violate the EMWA limits: (a) the process is unsteady or there was poor sample handling, (b) typographical error in the lab result, (c) online analyzer malfunction, (d) lab instrument or the process analyzer is no long passing statistical quality control checks, (e) the bias between the lab and analyzer naturally drifted apart over time.

When a violation of the EMWA High/Low limit occurs, the operator should respond by communicating with the laboratory and analyzer group to determine if there is an issue with the appropriate instruments or test results. The operator may also take an additional validation sample from the process to confirm a statistically significant bias shift has emerged. If a statistically significant bias shift is confirmed, the operator should contact the blending engineer to retune the structural bias correction function using the new lab and analyzer data.

If EWMA_{current} violates the EWMA HiHigh/LoLow limits, then the referee tests are considered an outlier and rejected. EWMA_{current} is not accepted and EWMAₚᵣᵢₒᵣ continues to be used in Eqn. 1. The above steps are repeated with the next validation sample.

When a violation of the EMWA HiHigh/LoLow limit occurs, the operator should respond by determining if there is an issue with the appropriate instruments or test results. The operator may also take an additional validation sample from the process to confirm a statistically significant bias shift has emerged.

Also, as discussed above, the present method can also be applied to mathematical models used to predict additive boost, such as from oxygenate. In such cases, the structural bias correction function of Eqn. 1 and Eqn. 3 (BCf( )) becomes an oxygenate boost function (EBf( )). The oxygenate boost function can be calculated via establishing an empirical relationship between a BOB hydrocarbon mixture and hydrocarbon mixture blended with oxygenate. Such relationships are discussed in U.S. Pat. Nos. 8,999,012, 8,986,402, and 8,322,200, which are incorporated by reference. The method remains the same except that the laboratory validation samples are taken and additive is added to the basestock. The method can also be applied to other products and associated additives, for example, distillate (bio diesel, cetane improver).

### Additional Embodiments

### Embodiment 1

A method for reducing bias between an online process analyzer measurement of a property of a hydrocarbon stream and a laboratory certification test result of the same property: obtaining a physical sample from the hydrocarbon stream at time t;

analyzing the physical sample via a laboratory certification test to obtain a laboratory certification test result for the property of the hydrocarbon stream; providing an online process analyzer to measure the property of the hydrocarbon stream; the online process analyzer obtaining a raw value of the property (“the raw analyzer output”); establishing an exponential-weighted moving average (EWMA) dynamic correction factor for the property measured by the online process analyzer, wherein establishing the EWMA dynamic correction factor comprises; collecting historical values of the property from a plurality of past laboratory certification tests; determining a structural bias correction function for the property by performing a regression analysis on the historical values; establishing a lambda weight, λ, between 0 and 1; calculating EWMA via the following equation:

EWMA_{current}=λ*(LRₜ−BCf(RAₜ))+(1−λ)*EWMAₚᵣᵢₒᵣ, where:
EWMA_{current}=Current exponential-weighted moving average dynamic correction factor;
λ=lambda weight;
LRₜ=the laboratory certification test result of the property of the physical sample at time t;
BCf(RAₜ)=the structural bias correction function applied to the raw analyzer output of the property at time t; and
EWMAₚᵣᵢₒᵣ=the most recently prior calculated EWMA;
   correcting the raw analyzer output via the following relationship:

CAₜ=BCf(RAₜ)+EWMA_{current}, where CAₜ is the Corrected Analyzer output at time t; and

comparing the Corrected Analyzer output at time t to the laboratory certification test result at time t to determine the bias between the two values.

### Embodiment 2

The method of embodiment 1, further comprising: establishing an EWMA high limit and an EWMA low limit via the following equation:

EWMA_{Hi/Low}=+/−k₁*Stdev of Residuals, where:
k₁=any number between 2 and 4;
Residuals=LRₜ−CAₜ; and
Stdev=Standard Deviation
   comparing the EWMA_{current} to EWMA_{Hi} and EWMA_{Low}; and adjusting the EWMA to the calculated value of EWMA_{current} if EWMA_{current} is between EWMA_{Hi} and EWMA_{Low}.

### Embodiment 3

The method of embodiment 2, wherein if EWMA_{current} is greater than EWMA_{high} or is less than the EWMA_{low} limit, then EWMA_{current} is set to the EWMA_{high} limit or the EWMA_{low} limit accordingly.

### Embodiment 4

The method of embodiments 2 or 3, further comprising:

establishing an EWMA hihigh limit and an EWMA blow limit via the following equation

EWMA_{HiHigh/LoLow}=+/−12*Stdev of Residuals, where:
k₂=any number between 5 and 8;
Residuals=LRₜ−CAₜ; and
Stdev=Standard Deviation

rejecting EWMA_{current} if EWMA_{current} violates the established EWMA_{HiHigh} limit or the established EWMA_{LoLow} limit and correcting the Raw Analyzer output using EWMAₚᵣᵢₒᵣ.

### Embodiment 5

The method of any of embodiments 2-4, wherein k₁=3.

### Embodiment 6

The method of embodiments 4 or 5, wherein k₂=6.

### Embodiment 7

The method of any of embodiments 4-6, wherein k₂=2*k₁

### Embodiment 8

The method of any of the previous embodiments, wherein lambda weight is 0.8 or greater.

### Embodiment 9

The method of any of the previous embodiments, wherein the property is Reed Vapor Pressure, TV/L(20), sulfur, aromatic content, olefin content, benzene content, distillation points, octane, API gravity, flash point, kinematic viscosity, cetane level, cloud point, or pour point.

### Embodiment 10

The method of any of the previous embodiments, wherein the hydrocarbon stream is a basestock for oxygenate blending (BOB).

### Embodiment 11

The method of any of the previous embodiments, wherein the hydrocarbon stream is a basestock for oxygenate blending (BOB); wherein the obtaining a physical sample from the hydrocarbon stream occurs before the hydrocarbon stream is blended with an oxygenate additive and the laboratory certification test occurs after the physical sample is blended with the oxygenate additive.

### Embodiment 12

The method of embodiment 11, wherein the oxygenate is ethanol.

### Example 1: Dynamic Bias Correction of TV/L (20) when Blending BOB Gasoline

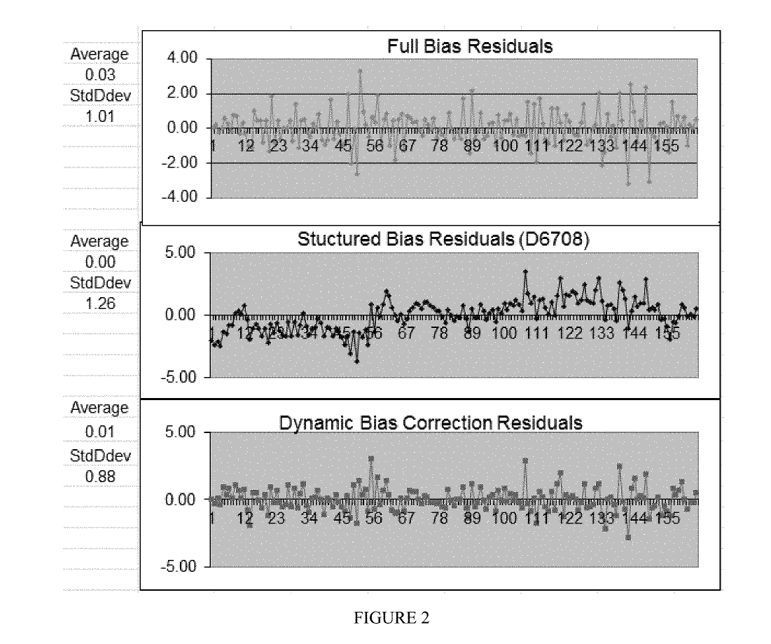

TV/L (20) is a measure of the temperature at which the vapor to liquid ratio of the hydrocarbon mixture is 20 to 1. In FIG. 2, Three different bias correction techniques and their associated residuals are compared using TV/L (20) data from a variety of blended BOB gasolines. The top chart shows residuals from full bias correction where: Residual=LR−(RA+Prior Residual). The middle chart shows residuals from structural bias correction function where: Residual=LR−BCf (RA). The bottom chart shows residuals from EWMA dynamic bias correction where: Residual=LR−CA. In each of the charts, the residuals—i.e. the difference between laboratory referee tests and bias corrected online process analyzers at the time the laboratory sample was taken—are plotted over time. The standard deviation of the residuals from full bias and structured bias correction are about 1° F. and 1.26° F. respectively. While full bias correction keeps the online analyzer results closely matched to the referee results, it does not reduce system noise and therefore cannot be used for improved control. The structured bias correction function (middle chart) reduces the standard deviation of the residuals for extended periods of time, however the residuals are not randomly distributed around zero, but rather they tend to remain above and below zero for periods of time, indicating the data is autocorrelated. The bottom chart shows that the standard deviation of the residuals for the present method is significantly improved (lower), as compared to the previous methods, and the residuals are randomly distributed around zero. Accordingly, the present method provides a superior bias correction in that it simultaneously minimizes bias and reduces variation, resulting in improved process control using the analyzer as a prediction of the laboratory referee test results.

### Example 2: Dynamic Bias Correction of TV/L (20) in Prediction of Ethanol Boost

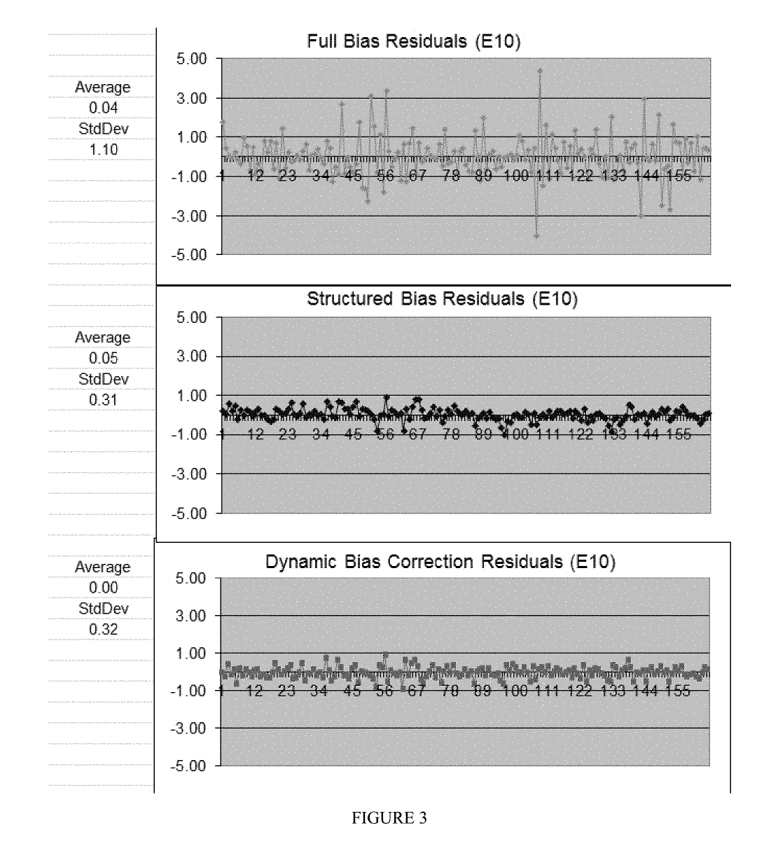

In FIG. 3, three different bias correction techniques and their associated residuals are compared using TV/L (20) data from a variety of blended E10 gasolines (10% by volume ethanol). The top chart shows residuals from full bias correction where: Residual=LR_E−(EBf (RA)+Prior Residual). The middle chart shows residuals from structural bias correction function where: Residual=LR_E−ECf (RA). The bottom chart shows residuals from EWMA dynamic bias correction where: Residual=LR_E−CA_E. In each of the charts, the residuals—i.e. the difference between laboratory referee tests and bias corrected online process analyzers at the time the laboratory sample was taken are plotted over time. Note that values from the laboratory test results are from validation samples already blended with ethanol, whereas values from online process analyzers are taken from basestocks and the oxygenate boost correction function is applied to predict the ethanol blended values. The standard deviation of the residuals from full bias (top chart) using the ethanol boost from a prior validation sample is 1.1° F. The standard deviation of residuals when using a linear ethanol boost function (middle chart) is 0.31° F., but is not centered around 0. The standard deviation of residuals when using the EWMA dynamic correction (bottom chart) is 0.32° F. and is centered around 0. The EWMA dynamic bias correction method provides a significant reduction in standard deviation compared to the other techniques, allowing for improved prediction properties of BOB blended with Ethanol.

1. A method for reducing bias between an online process analyzer measurement of a property of a hydrocarbon stream and a laboratory certification test result of the same property:
obtaining a physical sample from the hydrocarbon stream at time t;
analyzing the physical sample via a laboratory certification test to obtain a laboratory certification test result for the property of the hydrocarbon stream;
providing an online process analyzer to measure the property of the hydrocarbon stream; the online process analyzer obtaining a raw value of the property (“the raw analyzer output”);
establishing an exponential-weighted moving average (EWMA) dynamic correction factor for the property measured by the online process analyzer, wherein establishing the EWMA dynamic correction factor comprises;
collecting historical values of the property from a plurality of past laboratory certification tests;
determining a structural bias correction function for the property by performing a regression analysis on the historical values;
establishing a lambda weight, λ, between 0 and 1;
calculating EWMA via the following equation
EWMA_{current}=λ*(LRₜ−BCf(RAₜ))+(1−λ)*EWMAₚᵣᵢₒᵣ, where:
EWMA_{current}=Current exponential-weighted moving average dynamic correction factor;
λ=lambda weight;
LRₜ=the laboratory certification test result of the property of the physical sample at time t;
BCf(RAₜ)=the structural bias correction function applied to the raw analyzer output of the property at time t; and
EWMAₚᵣᵢₒᵣ=the most recently prior calculated EWMA;
correcting the raw analyzer output via the following relationship:
CAₜ=BCf(RAₜ)+EWMA_{current}, where CAₜ is the Corrected Analyzer output at time t; and
comparing the Corrected Analyzer output at time t to the laboratory certification test result at time t to determine the bias between the two values.

2. The method of claim 1, further comprising:
establishing an EWMA high limit and an EWMA low limit via the following equation:
EWMA_{Hi/Low}=+/−k₁*Stdev of Residuals, where:
k₁=any number between 2 and 4;
Residuals=LRₜ−CAₜ; and
Stdev=Standard Deviation
comparing the EWMA_{current} to EWMA_{Hi} and EWMA_{Low}; and
adjusting the EWMA to the calculated value of EWMA_{current} if EWMA_{current} is between EWMA_{Hi} and EWMA_{Low}.

3. The method of claim 2, wherein if EWMA_{current} is greater than EWMA_{high} or is less than the EWMA_{low} limit, then EWMA_{current} is set to the EWMA_{high} limit or the EWMA_{low} limit accordingly.

4. The method of claim 2, further comprising:
establishing an EWMA hihigh limit and an EWMA lolow limit via the following equation
EWMA_{HiHigh/LoLow}=+/−12*Stdev of Residuals, where:
k₂=any number between 5 and 8;
Residuals=LRₜ−CAₜ; and
Stdev=Standard Deviation
rejecting EWMA_{current} if EWMA_{current} violates the established EWMA_{HiHigh} limit or the established EWMA_{LoLow} limit and correcting the Raw Analyzer output using EWMAₚᵣᵢₒᵣ.

5. The method of claim 2, wherein k₁=3.

6. The method of claim 4, wherein k₂=6.

7. The method of claim 4, wherein k₂=2*k₁

8. The method of claim 1, wherein lambda weight is 0.8 or greater.

9. The method of claim 1, wherein the property is Reed Vapor Pressure, TV/L(20), sulfur, aromatic content, olefin content, benzene content, distillation points, octane, API gravity, flash point, kinematic viscosity, cetane level, cloud point, or pour point.

10. The method of claim 1, wherein the hydrocarbon stream is a basestock for oxygenate blending (BOB).

11. The method of claim 1, wherein the hydrocarbon stream is a basestock for oxygenate blending (BOB); wherein the obtaining a physical sample from the hydrocarbon stream occurs before the hydrocarbon stream is blended with an oxygenate additive and the laboratory certification test occurs after the physical sample is blended with the oxygenate additive.

12. The method of claim 11, wherein the oxygenate is ethanol.
